(19) United States
(12) Patent Application Publication
(10) Pub. No.: US 2026/0248741 A1
(43) Pub. Date: Aug. 27, 2026

(54) DEXMEDETOMIDINE-CONTAINING TRANSDERMAL PATCH AND PREPARATION METHOD THEREOF

(71) Applicant: Beijing Tide Pharmaceutical Co., Ltd., Beijing (CN)

(72) Inventors: Jianteng PAN, Beijing (CN); Shaopeng ZHONG, Beijing (CN); Yun WU, Beijing (CN); Pingping ZHANG, Beijing (CN); Zhiyang YUE, Beijing (CN); Lei LIU, Beijing (CN); Leling GAN, Beijing (CN)

(73) Assignee: Beijing Tide Pharmaceutical Co., Ltd., Beijing (CN)

(21) Appl. No.: 19/386,649

(22) Filed: Nov. 12, 2025

(30) Foreign Application Priority Data
Feb. 26, 2025 (CN) 202510214726.3

(51) Int. Cl. A61K 9/70; A61K 31/4174; A61K 31/55; A61K 47/10; A61K 47/14; A61K 47/32

(52) U.S. Cl. CPC A61K 9/7061; A61K 31/4174; A61K 31/55; A61K 47/10; A61K 47/14; A61K 47/32

(57) ABSTRACT

The present application relates to the field of pharmaceutical biotechnology, and specifically relates to a dexmedetomidine-containing transdermal patch. By adding a penetration enhancer, the transdermal rate and transdermal amount are increased, and the onset time is shortened; the patch content is reduced, the residual amount in the skin is lowered, and skin redness/scarring at the administration site caused by drug accumulation in the skin is effectively avoided.

### CROSS REFERENCE TO RELATED APPLICATION

This application claims the benefit of priority from Chinese Patent Application No. 202510214726.3, filed Feb. 26, 2025, the contents of which are incorporated herein by reference.

### TECHNICAL FIELD

The present application relates to the field of pharmaceutical biotechnology, and specifically relates to a dexmedetomidine-containing transdermal patch and preparation method thereof.

### BACKGROUND ART

Insomnia is the most common sleep disorder, specifically manifested as difficulty falling asleep, sleep maintenance disturbances, early awakening, decreased sleep quality, and reduced total sleep time (usually less than 6 hours), accompanied by daytime dysfunction. Sleep has a significant impact on human health, and chronic insomnia can lead to weakened immunity, anxiety and other health problems. Sleep disorders have become a huge hidden which threats to people's health.

Dexmedetomidine, as a highly selective ADAR2 agonist, can inhibit transmission of norepinephrinergic nerve derived from the locus coeruleus to the ventrolateral preoptic nucleus (VLPO), thereby disinhibiting VLPO and triggering suppression of cortical arousal nuclei, resulting in sedative and hypnotic effects. Compared with the reduction or no significant effect of the currently commonly used drugs on stage 3 of non-rapid eye movement (NREM) sleep, dexmedetomidine can prolong total sleep time, improve sleep efficiency, enhance sleep architecture (particularly prolonging stage 3 of NREM sleep), and treat insomnia disorders. In recent years, formulations for the treatment of insomnia through percutaneous absorption have become a hot topic for research and development. Dexmedetomidine can be administered via skin patches, with the active ingredient absorbed through the skin and entering the blood circulation throughout the body, offering convenience in terms of administration and good patient compliance.

The drug absorption process of transdermal patches is as follows: after application to the skin surface, the drug is first released from the formulation to the skin surface, then partitions into the stratum corneum, diffuses through the stratum corneum to the interface of the viable epidermis, partitions into the aqueous viable epidermis, continues diffusing to the dermis, and is absorbed by capillaries into blood circulation to exert its pharmacological effects.

The results of non-clinical studies of conventional dexmedetomidine transdermal patches show a long time to peak concentration and slow absorption rate. Clinically, drugs for insomnia treatment are expected to take effect rapidly within 30 minutes to 1 hour. Therefore, it is necessary to develop transdermal patches that can improve the absorption rate of dexmedetomidine.

CN116098876A discloses a dexmedetomidine transdermal patch, its preparation method, and application;

CN117582424A discloses a dexmedetomidine transdermal composition, transdermal patch, and their preparation methods and applications;

CN117959268A discloses a lipid-soluble sedative-hypnotic sleep aid patch for insomnia patients and its preparation method.

The drug penetration rates of the formulations in the above three patents are relatively slow, with long time to peak concentration in pig patch tests. The drug is not detected until after 4 hours in human patch test, indicating a slow onset of action. Additionally, drug accumulation in the skin causes redness/scarring at the administration site.

Clinically, insomnia treatment requires rapid onset within 1 hour, which is extremely unfavorable for the treatment of patients with insomnia, as they need to apply the patch a long time in advance. If the advance application is forgotten, it will affect the treatment of patients with insomnia. Therefore, it is necessary to address the slow onset of conventional patches, improve drug penetration rates, enable insomnia patients to fall asleep quickly, while avoiding skin redness/scarring at the administration site.

### SUMMARY

In view of the above technical status, the present application provides a dexmedetomidine-containing transdermal patch. The transdermal patch comprises a penetration enhancer, and the penetration enhancer is selected from dimethyl sulfoxide, azone, isopropyl palmitate, isopropyl myristate, oleic acid, ethyl oleate, oleyl alcohol, propylene glycol, polyglyceryl oleate, diethylene glycol monoethyl ether, eucalyptol, menthol, or a combination of two or more thereof. As one of the embodiments, preferably azone.

In the present application, as one of the embodiments, the amount of the penetration enhancer in the transdermal patch is 3%-30% by mass basis, preferably 5%-20%. As exemplary illustration, the amount of the penetration enhancer may be 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%.

In the present application, as one of the embodiments, the amount of dexmedetomidine in the transdermal patch is 4%-10% by mass fraction, preferably 4%-8%. As exemplary illustration, it may be 4%, 5%, 6%, 7%, 8%, 9%, or 10%.

In the present application, as one of the embodiments, the transdermal patch further comprises a crystallization inhibitor, and the crystallization inhibitor is selected from povidone. As one of the embodiments, preferably povidone K25, povidone K17, povidone K12, povidone K30, povidone K90, or copovidone VA64. As one of the embodiments, further preferably povidone K30.

In the present application, as one of the embodiments, the amount of crystallization inhibitor in the transdermal patch is 2%-12% by mass fraction, preferably 3%-8%. As exemplary illustration, it may be 2%, 3%, 4%, 5%, 6%, 7%, 8%, 9%, 10%, 11%, or 12%.

In the present application, as one of the embodiments, the transdermal patch further comprises an adhesive, and the adhesive is selected from acrylate pressure-sensitive adhesives.

In the present application, as one of the embodiments, the pressure-sensitive adhesive is an acrylate pressure-sensitive adhesive, preferably DURO-TAK 87-900A, DURO-TAK 87-9301, DURO-TAK 87-4098, DURO-TAK 387-2510, DURO-TAK 387-2287, DURO-TAK 87-4287, DURO-TAK 387-2516, DURO-TAK 87-2074, DURO-TAK 87-235A, DURO-TAK 387-2353, DURO-TAK 387-2852, DURO-TAK 387-2051, DURO-TAK 387-2052, DURO-TAK 387-2054, DURO-TAK 87-2194, DURO-TAK 87-2196, DURO-TAK 87-2677, GELVA GMS 9073, GELVA GMS 788, GELVA GMS 3253, or GELVA GMS 3083, or a combination of two or more thereof. As one of the embodiments, more preferably DURO-TAK 387-2516.

In the present application, as one of the embodiments, the amount of pressure-sensitive adhesive in the transdermal patch is 48%-94% by mass fraction, preferably 74%-88%. As exemplary illustration, the pressure-sensitive adhesive in the transdermal patch may be 75%, 76%, 77%, 78%, 79%, 80%, 81%, 82%, 83%, 84%, 85%, 86%, or 87%.

In the present application, as one of the embodiments, the transdermal patch comprises:
the amount of drug: 4%-10%;
the amount of penetration enhancer: 3%-30%;
the amount of povidone: 2%-12%; and
the amount of adhesive: 48%-94%.

In the present application, as one of the embodiments, the transdermal patch comprises:
the amount of drug: 4%-8%;
the amount of penetration enhancer: 5%-20%;
the amount of povidone: 3%-8%; and
the amount of adhesive: 74%-88%.

In the present application, as one of the embodiments, the transdermal patch further comprises:

| Component | wt % |
|---|---|
| Dexmedetomidine | 4.0 |
| Povidone K30 | 3.0 and |
| DURO-TAK 387-2516 | 93.0; |
| Dexmedetomidine | 4.0 |
| Povidone K30 | 3.0 |
| Azone | 10.0 and |
| DURO-TAK 387-2516 | 83.0; |
| Dexmedetomidine | 4.0 |
| Povidone K30 | 3.0 |
| Oleic acid | 10.0 and |
| DURO-TAK 387-2516 | 83.0; |
| Dexmedetomidine | 4.0 |
| Povidone K30 | 3.0 |
| Propylene glycol | 10.0 and |
| DURO-TAK 387-2516 | 83.0; |
| Dexmedetomidine | 4.0 |
| Povidone K30 | 3.0 |
| Diethylene glycol monoethyl ether | 10.0 and |
| DURO-TAK 387-2516 | 83.0; |
| Dexmedetomidine | 4.0 |
| Povidone K30 | 3.0 |
| Polyglyceryl oleate | 10.0 and |
| DURO-TAK 387-2516 | 83.0. |

In the present application, as one of the embodiments, the transdermal patch further comprises a backing layer.

In the present application, as one of the embodiments, the material of the backing layer is selected from polyester, polyethylene polyvinyl acetate resin, copolymer of polyethylene and vinyl acetate, polyvinyl chloride, or polyurethane, preferably ScotchPak™ 1109 or Cotran™ 9720.

In the present application, as one of the embodiments, the transdermal patch further comprises a protective layer.

In the present application, as one of the embodiments, the material of the protective layer is selected from Scotchpak™ 9744 release film or Scotchpak™ 9709 release film, preferably Scotchpak™ 9709 release film.

The present application further provides a preparation method for the dexmedetomidine-containing transdermal patch, characterized in that, the method comprising the following steps:
(1) dissolving dexmedetomidine and povidone in an organic solvent at 2-10 times the weight of dexmedetomidine, and adding the penetration enhancer, and mixing uniformly to obtain the drug solution;
(2) mix the drug solution obtained in step (1) with the adhesive, and stirring uniformly to obtain the adhesive mixed solution;
(3) after defoaming, coating the adhesive mixed solution onto the release film, and drying to remove the organic solvent; and
(4) applying the backing layer and cutting to obtain the transdermal patch.

In the present application, as one of the embodiments, the method further comprises:
(1) dissolving the prescribed amounts of dexmedetomidine and povidone K30 in organic solvent, and adding the penetration enhancer, and mixing uniformly to obtain the drug solution;
(2) mixing the drug solution obtained in step (1) with the adhesive at a ratio of 1:1-10, and stirring uniformly to obtain the adhesive mixed solution. As exemplary illustration, the ratio of the drug solution to the adhesive may be 1:1, 1:2, 1:3, 1:4, 1:5, 1:6, 1:7, 1:8, 1:9, or 1:10;
(3) after standing for defoaming, coating the adhesive mixed solution onto the release film with a controlled thickness of 0.22 mm, and drying at 90° C. under forced air for 20 min to remove the organic solvent;
(4) applying the backing layer and cutting to obtain the transdermal patch.

In the present application, as one of the embodiments, the amount of organic solvent in the transdermal patch is 5-10 times the weight of dexmedetomidine by mass fraction. As exemplary illustration, it may be 5 times, 6 times, 7 times, 7.5 times, 8 times, 9 times, or 10 times.

In the present application, as one of the embodiments, the organic solvent is selected from ethanol, ethyl acetate, n-heptane, or dimethyl sulfoxide, preferably ethanol.

According to investigation, the transdermal patch of the present application improves the transdermal rate and penetration amount by incorporating penetration enhancer, shortens the onset time, reduces patch content and skin residue, shortens application time, and effectively avoids skin redness/scarring at the administration site caused by drug accumulation in the skin.

Advantages of the present application include:
(1) excellent adhesion performance, achieved by the pressure-sensitive adhesive;
(2) fast transdermal rate and high penetration amount;
(3) reduced drug concentration and skin residue, with no significant skin irritation; and
(4) good stability.

The present application has been validated through in vitro transdermal experiments, animal pharmacokinetics, and skin irritation tests, demonstrating good adhesion performance, fast transdermal rate, high penetration amount, minimal skin irritation, and sleep-aiding effects.

### BRIEF DESCRIPTION OF THE DRAWINGS

FIG. 1: The change curve of cumulative transdermal penetration amount over time in the in vitro transdermal experiment using 30-day-old Bama miniature pig skin.

FIG. 2: The drug concentration-time curves of Formulations 12-17.

### DETAILED DESCRIPTION

The following examples are provided to further illustrate the present application but are not intended to limit the effective scope of the invention in any way.

### Example 1—Screening of Solvent Type and Amount

The dexmedetomidine transdermal patch of the present application was proposed to be prepared using a solvent method, wherein the materials used in the formulation, dexmedetomidine and povidone were solids, while other materials are liquids. To ensure uniform mixing of materials, solvents were required to dissolve the solid materials before mixing. Commonly used solvents included methanol, ethanol, isopropanol, ethyl acetate, n-heptane, and dimethyl sulfoxide, preferably methanol, ethanol, isopropanol, or dimethyl sulfoxide. According to the requirements of the solvent method process, the solvent must ultimately be removed by drying, so the boiling point should be relatively low, such as methanol, ethanol, and isopropanol, preferably ethanol. The amount of ethanol was determined by adding gradually while observing the dissolution state of the solid materials. The results are shown in Tables 1 and 2:

TABLE 1

| Solvent | Boiling Point | Material Dissolution Status |
|---|---|---|
| Ethanol | 78.4° C. | Completely dissolved |
| Ethyl acetate | 77.2° C. | Not completely dissolved |
| n-Heptane | 98.4° C. | Not completely dissolved |
| Dimethyl sulfoxide | 189° C. | Completely dissolved |

TABLE 2

| The amount of ethanol | Dissolution State of Solid Materials |
|---|---|
| 1 time the weight of | Solid material surface not fully wetted |
| Dexmedetomidine | |
| 2 times the weight of | Solid material surface fully wetted; |
| Dexmedetomidine | povidone is viscous and dissolves slowly |
| 5 times the weight of | Solid material surface fully wetted; |
| Dexmedetomidine | complete dissolution after 30 min of |
| | magnetic stirring |
| 10 times the weight of | Solid material surface fully wetted; |
| Dexmedetomidine | complete dissolution after 10 min of |
| | magnetic stirring |
| 7.5 times the weight of | Solid material surface fully wetted; |
| Dexmedetomidine | complete dissolution after 10 min of |
| | magnetic stirring |
| 7 times the weight of | Solid material surface fully wetted; |
| Dexmedetomidine | minor undissolved residue after 10 min |
| | of magnetic stirring |

The results show that the materials can be completely dissolved in ethanol and dimethyl sulfoxide. Ethanol has a relatively lower boiling point. Considering the solubility of the materials and process requirements, the solvent is preferably ethanol. When the amount of ethanol is 2 times the weight of dexmedetomidine, the solid material surface is fully wetted, but povidone is viscous and forms a sticky layer after surface wetting, making it difficult for the solvent to penetrate into the material, resulting in slow dissolution that requires prolonged stirring for complete dissolution. When the amount of ethanol is 5 times the weight of dexmedetomidine, 30 min of stirring is required for complete dissolution. When the amount of ethanol is 7.5 times or more the weight of dexmedetomidine, complete dissolution is achieved after 10 min of stirring. Therefore, the amount of ethanol is preferably 7.5 times the weight of dexmedetomidine. The amount of solvent is preferably more than 5 times that of dexmedetomidine, including 5 times, and further preferably more than 7.5 times that of dexmedetomidine, including 7.5 times.

### Example 2—Comparison of Different Adhesives

DURO-TAK 387-2516 (with ethyl acetate as solvent, solid content of 41.5%, containing hydroxyl functional groups) and DURO-TAK 387-2074 (with ethyl acetate as solvent, solid content of 29.5%, containing hydroxyl and carboxyl functional groups) were selected. The formulations are shown in Table 3:

TABLE 3

wt % (calculated based on solids)

| Component | Formulation 1 | Formulation 2 |
|---|---|---|
| Dexmedetomidine | 10 | 10 |
| Povidone K30 | 5 | 5 |
| Ethanol | / | / |
| DURO-TAK 387-2516 | 85 | / |
| DURO-TAK 387-2074 | / | 85 |

Note: Ethanol was used as the solvent and is ultimately removed. The amount of ethanol was 7.5 times the weight of dexmedetomidine.

The prescribed amounts of dexmedetomidine and povidone K30 were dissolved in ethanol, and then the pressure-sensitive adhesive was added and uniformly mixed. After standing for defoaming, the mixture was coated onto a release film with a controlled thickness of 0.22 mm, followed by drying at 90° C. under forced air for 20 min. Finally, a backing layer was applied, and the patch was cut into 2.5 cm² pieces.

The average content of Formulation 1 was 1.14 mg/patch, and the average content of Formulation 2 was 1.09 mg/patch. To compare the actual skin penetration of the patches, one patch from each formulation was applied to the outer side of upper arms of 6 healthy volunteers. After 2 hours, the patches were removed, and the adhesion status on the skin was observed. The drug residue was analyzed. The transdermal rate was calculated as (patch content-residue amount)/patch content. The results were shown in Table 4:

TABLE 4

| | Formulation 1 | | Formulation 2 | |
|---|---|---|---|---|
| | 2 h | | 2 h | |
| | Transdermal | | Transdermal | |
| | Rate (%) | Adhesion Status | Rate (%) | Adhesion Status |
| Volunteer 1 | 6.5 | Good adhesion, | 7.4 | Good adhesion, |
| | | no curled edges | | no curled edges |
| Volunteer 2 | 9.9 | Good adhesion, | 5.5 | Good adhesion, |
| | | no curled edges | | no curled edges |
| Volunteer 3 | 8.0 | Good adhesion, | 4.1 | Slightly curled |
| | | no curled edges | | edge |
| Volunteer 4 | 8.6 | Good adhesion, | 6.2 | Good adhesion, |
| | | no curled edges | | no curled edges |
| Volunteer 5 | 7.1 | Good adhesion, | 4.3 | Slightly curled |
| | | no curled edges | | edge |
| Volunteer 6 | 15.1 | Good adhesion, | 6.0 | Good adhesion, |
| | | no curled edges | | no curled edges |
| Average | 9.2 | / | 5.6 | / |
| Transdermal Rate | | | | |
| Average | 21.0 μg/cm²/h | / | 12.2 μg/cm²/h | / |
| Transdermal Rate | | | | |

From the results, it can be seen that DURO-TAK 387-2516 as adhesive provided good adhesion and relatively higher transdermal rates. Since two patches were applied to each volunteer simultaneously, and the 2-hour penetration amount was relatively large, all of which are greater than 110 μg, therefore most volunteers (4 cases) reported varying degrees of drowsiness after the patch was removed.

Additionally, no adverse reactions such as pain, swelling, or itching were reported from volunteers, indicating good safety of the patches. However, due to the high drug content in the patches, mild skin irritation was observed, resulting in patch-induced scarring in half of the volunteers (3 cases), which disappeared within approximately 2 days.

### Example 3—Comparison of Penetration Enhancer

Penetration enhancer such as azone, oleic acid, propylene glycol, diethylene glycol monoethyl ether, and polyglyceryl oleate were selected and compared with a sample without penetration enhancer. The formulations were shown in Table 5:

TABLE 5

| | wt % (calculated based on solid content) | | | | | |
|---|---|---|---|---|---|---|
| | Formulation | Formulation | Formulation | Formulation | Formulation | Formulation |
| Component | 12 | 13 | 14 | 15 | 16 | 17 |
| Dexmedetomidine | 4.0 | 4.0 | 4.0 | 4.0 | 4.0 | 4.0 |
| Povidone K30 | 3.0 | 3.0 | 3.0 | 3.0 | 3.0 | 3.0 |
| Ethanol | / | / | / | / | / | / |
| Azone | / | 10.0 | / | / | / | / |
| Oleic acid | / | / | 10.0 | / | / | / |
| Propylene glycol | / | / | / | 10.0 | / | / |
| Diethylene glycol | / | / | / | / | 10.0 | / |
| monoethyl ether | | | | | | |
| Polyglycerol | / | / | / | / | / | 10.0 |
| Oleate | | | | | | |
| DURO-TAK | 93.0 | 83.0 | 83.0 | 83.0 | 83.0 | 83.0 |
| 387-2516 | | | | | | |

Note: Ethanol was used as the solvent, with an amount of 7.5 times the weight of dexmedetomidine, and was ultimately removed by drying.

The prescribed amounts of dexmedetomidine and povidone K30 were dissolved in ethanol, mixed with the penetration enhancer, and then uniformly mixed after added with the pressure-sensitive adhesive. After standing for defoaming, the mixture was coated onto a release film with a controlled thickness of 0.18 mm, followed by drying at 90° C. under forced air for 10 min. Finally, a backing layer was applied, and the patch was cut into 10 cm² square pieces.

### Example 4—In Vitro Transdermal Experiment

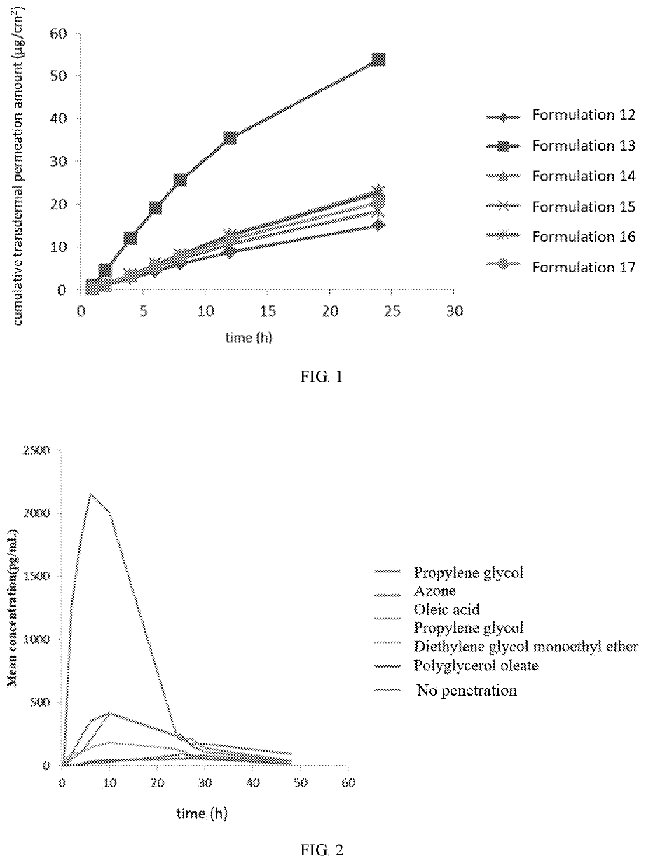

Thirty-day-old Bama miniature pig skin was used as the transdermal material for the in vitro transdermal experiment using Franz vertical diffusion cells. The receiving solution was phosphate buffer (pH=7.40), with a bath temperature of 32° C.±0.5° C. and a stirring speed of 500 rpm. Four parallel samples are prepared and samples were taken at 1 h, 2 h, 4 h, 6 h, 8 h, 12 h, and 24 h, respectively, as test solutions, while blank receiving solution was replenished. The results were shown in Table 6 and FIG. 1:

TABLE 6

| | | Cumulative Penetration Amount (μg/cm²) | | | | | |
|---|---|---|---|---|---|---|---|
| | | Formulation | Formulation | Formulation | Formulation | Formulation | Formulation |
| Time (h) | | 12 | 13 | 14 | 15 | 16 | 17 |
| 1 | h | 0.3 | 1.0 | 0.3 | 0.3 | 0.4 | 0.4 |
| 2 | h | 1.0 | 4.5 | 1.0 | 1.2 | 1.2 | 1.2 |
| 4 | h | 2.6 | 12.0 | 3.2 | 3.4 | 3.1 | 3.3 |
| 6 | h | 4.3 | 19.1 | 5.7 | 5.9 | 5.2 | 5.5 |
| 8 | h | 6.0 | 25.6 | 8.2 | 8.2 | 7.1 | 7.8 |
| 12 | h | 8.7 | 35.4 | 12.7 | 12.5 | 10.5 | 11.6 |
| 24 | h | 15.0 | 53.9 | 23.2 | 22.7 | 18.4 | 20.5 |

| | | | | | | |
|---|---|---|---|---|---|---|
| 24 h Penetration | 9.8 | 34.6 | 15.0 | 14.8 | 12.0 | 13.5 |
| amount (%) | | | | | | |
| Sample | 82.1 | 60.2 | 77.6 | 78.7 | 80.4 | 78.9 |
| residue | | | | | | |
| Intradermal | 5.5 | 3.4 | 5.0 | 4.9 | 5.4 | 5.6 |
| residue | | | | | | |
| Mass | 97.4 | 98.2 | 97.6 | 98.4 | 97.8 | 98.0 |
| balance | | | | | | |

From the results, it can be seen that after the addition of penetration enhancer, the in vitro transdermal rate and penetration amount were increased to varying degrees. Among them, the patch with azone shows the most significant increase in transdermal rate and penetration amount, with relatively less intradermal residue and high safety.

### Example 5—Screening of Penetration Enhancer Amount

Since the patch with azone shows the most significant increase in transdermal rate and penetration amount, azone was selected as the penetration enhancer for dosage screening. The formulations were shown in Table 7:

TABLE 7

| | wt % (calculated based on solid content) | | | | |
|---|---|---|---|---|---|
| Component | Formulation 12 | Formulation 13 | Formulation 18 | Formulation 19 | Formulation 20 |
| Dexmedetomidine | 4.0 | 4.0 | 4.0 | 4.0 | 4.0 |
| Povidone K30 | 3.0 | 3.0 | 3.0 | 3.0 | 3.0 |
| Ethanol | / | / | / | / | / |
| Azone | / | 10.0 | 5.0 | 15.0 | 30.0 |
| DURO-TAK 387-2516 | 93.0 | 83.0 | 88.0 | 78.0 | 63.0 |

Note: Ethanol was used as the solvent, with an amount of 7.5 times the weight of dexmedetomidine, and was ultimately removed by drying.

The prescribed amounts of dexmedetomidine and povidone K30 were dissolved in ethanol, mixed with the penetration enhancer, and then uniformly mixed after added with the pressure-sensitive adhesive. After standing for defoaming, the mixture was coated onto a release film with a controlled thickness of 0.18 mm, followed by drying at 90° C. under forced air for 10 min. Finally, a backing was applied, and the patch was cut into 10 cm² square pieces. The results were shown in Table 8:

TABLE 8

| | Cumulative Penetration Amount (μg/cm²) | | | | |
|---|---|---|---|---|---|
| Time (h) | Formulation 12 | Formulation 13 | Formulation 18 | Formulation 19 | Formulation 20 |
| 1 h | 0.3 | 1.0 | 0.3 | 0.3 | 0.4 |
| 2 h | 1.0 | 4.5 | 1.0 | 4.1 | 5.2 |
| 4 h | 2.6 | 12.0 | 3.5 | 13.4 | 13.1 |
| 6 h | 4.3 | 19.1 | 4.7 | 18.9 | 19.2 |
| 8 h | 6.0 | 25.6 | 7.2 | 26.2 | 27.1 |
| 12 h | 8.7 | 35.4 | 9.7 | 35.5 | 36.5 |
| 24 h | 15.0 | 53.9 | 16.2 | 52.7 | 55.4 |

The results show that when the amount of azone was 5%, no significant increase in transdermal rate or penetration amount was observed. When the amount of azone was 10%, the transdermal rate and penetration amount increase significantly. Further increasing the amount of azone to 15% and 30% does not show significant improvement compared to 10%. Therefore, the amount of azone was preferably 10%.

### Example 6—Pharmacokinetic Study

Six 4-month-old male Bama miniature pigs were selected and divided into 6 groups corresponding to Formulations 12-17. One patch was applied to the depilated skin of each Bama miniature pig. Blood samples were collected 10 min before administration and at 0.5 h, 1 h, 2 h, 4 h, 6 h, 10 h, 24 h, 27h, 30h, and 48 h after administration. The plasma drug concentration at each time point was determined using LC-MS/MS. The study was conducted in 3 rounds with animals replaced each time and a washout period of 5-7 days. The drug concentration-time curves of Formulations 12-17 were plotted as shown in FIG. 2. The results show that the blood drug concentration of the patches with azone added rose rapidly and higher.

### Example 7—Skin Irritation Test

Formulation 13 from Example 3 was cut into 2.5 cm² size. One patch was respectively applied to the inner side of upper arms of each of 6 healthy volunteers. The patches adhere well to the skin. After 3 hours, the patches were removed, and no obvious skin scars were observed. All volunteers reported varying degrees of drowsiness.

### Example 8—Stability Test

Samples of Formulation 13 from Example 3 were placed under accelerated conditions (40° C., 75% RH) for 3 months, and changes in related substances were analyzed.

### Chromatographic Conditions:

Detector: UV detector (detection wavelength: 210 nm)
Column: Waters X-Bridge C18 (4.6×250 mm, 5 μm)
Column temperature: 35° C.
Flow rate: 0.8 mL/min
Injection volume: 100 μL
Total analysis time: 40 min
Autosampler: Room temperature

Mobile phase: A: 3.2 g of disodium hydrogen phosphate dodecahydrate and 0.12 g of anhydrous sodium dihydrogen phosphate were dissolved in 1000 mL of water, and the pH was adjusted to 5.50 with phosphoric acid; B: Methanol; Ratio: A: B=50:50.

Results of Related Substances are shown in Table 9:

TABLE 9

| | | Maximum | |
|---|---|---|---|
| | | Single | Total |
| Time | Content % | Impurity % | Impurities % |
| Day 0 | 101.7 | Not detected | Not detected |
| One Month | 101.0 | 0.04 | 0.09 |
| Two Months | 101.6 | 0.04 | 0.27 |
| Three Months | 100.7 | 0.05 | 0.34 |

The results indicate that after 3 months under accelerated conditions, no significant changes in the active ingredient or related substances were observed.

What is claimed is:

1. A dexmedetomidine-containing transdermal patch, wherein the transdermal patch comprises a penetration enhancer, and the penetration enhancer is selected from the group consisting of dimethyl sulfoxide, azone, isopropyl palmitate, isopropyl myristate, oleic acid, ethyl oleate, oleyl alcohol, propylene glycol, polyglyceryl oleate, diethylene glycol monoethyl ether, eucalyptol, menthol and combinations of two or more thereof.

2. The transdermal patch according to claim 1, wherein the penetration enhancer is azone.

3. The transdermal patch according to claim 1, wherein the amount of the penetration enhancer in the transdermal patch is 3%-30% by mass fraction.

4. The transdermal patch according to claim 3, wherein the amount of the penetration enhancer in the transdermal patch is 5%-20% by mass fraction.

5. The transdermal patch according to claim 1, wherein the amount of dexmedetomidine in the transdermal patch is 4%-10% by mass fraction.

6. The transdermal patch according to claim 5, wherein the amount of dexmedetomidine in the transdermal patch is 4%-8%.

7. The transdermal patch according to claim 1, wherein the transdermal patch further comprises a crystallization inhibitor, and the crystallization inhibitor is povidone.

8. The transdermal patch according to claim 7, wherein the povidone is selected from the group consisting of povidone K25, povidone K17, povidone K12, povidone K30, povidone K90, and copovidone VA64.

9. The transdermal patch according to claim 8, wherein the povidone is povidone K30.

10. The transdermal patch according to claim 1, wherein the transdermal patch further comprises an adhesive, and the adhesive is an acrylate pressure-sensitive adhesive.

11. The transdermal patch according to claim 10, wherein the acrylate pressure-sensitive adhesive is selected from the group consisting of DURO-TAK 87-900A, DURO-TAK 87-9301, DURO-TAK 87-4098, DURO-TAK 387-2510, DURO-TAK 387-2287, DURO-TAK 87-4287, DURO-TAK 387-2516, DURO-TAK 87-2074, DURO-TAK 87-235A, DURO-TAK 387-2353, DURO-TAK 387-2852, DURO-TAK 387-2051, DURO-TAK 387-2052, DURO-TAK 387-2054, DURO-TAK 87-2194, DURO-TAK 87-2196, DURO-TAK 87-2677, GELVA GMS 9073, GELVA GMS 788, GELVA GMS 3253, GELVA GMS 3083, and combinations of two or more thereof.

12. The transdermal patch according to claim 10, wherein the content of the pressure-sensitive adhesive in the transdermal patch is 48%-94% by mass basis.

13. The transdermal patch according to claim 10, wherein the content of the pressure-sensitive adhesive in the transdermal patch is 74%-88% by mass basis.

14. The transdermal patch according to claim 1, wherein the transdermal patch comprises:
the amount of drug: 4%-10%;
the amount of penetration enhancer: 3%-30%;
the amount of povidone: 2%-12%; and
the amount of adhesive: 48%-94%.

15. The transdermal patch according to claim 1, wherein the transdermal patch comprises:
the amount of drug: 4%-8%;
the amount of penetration enhancer: 5%-20%;
the amount of povidone: 3%-8%; and
the amount of adhesive: 74%-88%.

16. The transdermal patch according to claim 1, wherein the transdermal patch further comprises one of:

| Component | wt % |
|---|---|
| Dexmedetomidine | 4.0, |
| Povidone K30 | 3.0, and |
| DURO-TAK 387-2516 | 93.0; |
| Dexmedetomidine | 4.0, |
| Povidone K30 | 3.0, |
| Azone | 10.0, and |
| DURO-TAK 387-2516 | 83.0; |
| Dexmedetomidine | 4.0, |
| Povidone K30 | 3.0, |
| Oleic acid | 10.0, and |
| DURO-TAK 387-2516 | 83.0; |
| Dexmedetomidine | 4.0, |
| Povidone K30 | 3.0, |
| Propylene glycol | 10.0, and |
| DURO-TAK 387-2516 | 83.0; |
| Dexmedetomidine | 4.0, |
| Povidone K30 | 3.0, |
| Diethylene glycol monoethyl ether | 10.0, and |
| DURO-TAK 387-2516 | 83.0; |
| or | |
| Dexmedetomidine | 4.0, |
| Povidone K30 | 3.0, |
| Polyglyceryl oleate | 10.0, and |
| DURO-TAK 387-2516 | 83.0. |

17. The transdermal patch according to claim 1, wherein the transdermal patch further comprises a backing layer, and wherein the material of the backing layer is selected from polyester, polyethylene polyvinyl acetate resin, copolymer of polyethylene and vinyl acetate, polyvinyl chloride, polyurethane, preferably ScotchPak™1109 or Cotran™9720, and
wherein the transdermal patch further comprises a protective layer, wherein the material of the protective layer is selected from Scotchpak™9744 release film or Scotchpak™9709 release film, preferably Scotchpak™9709 release film.

18. A method of preparing the transdermal patch according to claim 1, comprising the following steps:
(1) dissolving dexmedetomidine and povidone in an organic solvent at 2-10 times the weight of dexmedetomidine, and adding the penetration enhancer, and mixing uniformly to obtain the drug solution;
(2) mixing the drug solution obtained in step (1) with the adhesive, and stirring uniformly to obtain the adhesive mixed solution;
(3) after defoaming, coating the adhesive mixed solution onto the release film, and drying to remove the organic solvent; and
(4) applying the backing layer and cutting to obtain the transdermal patch, preferably, the method comprises:
(1) dissolving the prescribed amount of dexmedetomidine and povidone K30 in organic solvent, and adding the penetration enhancer, and mixing uniformly to obtain the drug solution;
(2) mixing the drug solution obtained in step (1) with the adhesive at a ratio of 1:1-10, and stirring uniformly to obtain the adhesive mixed solution;
(3) after standing for defoaming, coating the adhesive mixed solution onto the release film with a controlled thickness of 0.22 mm, and then drying at 90° C. with air blowing for 20 min. to remove the organic solvent; and
(4) applying the backing layer and cutting to obtain the transdermal patch.

19. The method according to claim 18, wherein the amount of the organic solvent is 5-10 times the weight of dexmedetomidine by mass fraction.

20. The method according to claim 18, wherein the organic solvent is selected from ethanol, ethyl acetate, n-heptane, dimethyl sulfoxide, preferably ethanol.
